(19) United States
(12) Patent Application Publication
(10) Pub. No.: US 2021/0345961 A1
(43) Pub. Date: Nov. 11, 2021

(54) WEARABLE PHYSIOLOGIC STATE MONITORING DEVICE

(71) Applicant: SOFT SENSE LIFE TECH PTE. LTD., Singapore (SG)

(72) Inventor: KUAN-CHIEN CHOU, Taipei City (TW)

(21) Appl. No.: 17/308,236

(22) Filed: May 5, 2021

(30) Foreign Application Priority Data
May 9, 2020 (CN) 202010385309.2

(51) Int. Cl. A61B 5/00; H05K 1/02; H05K 1/09; H05K 1/03; A61B 5/01; A61B 5/28

(52) U.S. Cl. CPC A61B 5/6804; H05K 1/028; H05K 1/092; H05K 1/0393; A61B 2562/0215; A61B 5/28; H05K 2201/10151; A61B 2562/0261; A61B 5/01

(57) ABSTRACT

A wearable physiologic state monitoring device includes a textile fabric, a flexible sensing unit, and a control unit. The textile fabric has a first surface and a second surface opposite to each other. The flexible sensing unit is joined to the first surface of the textile fabric and has a flexible substrate and a sensing element. The flexible substrate has a bearing surface, and a patterned conductive circuit is provided on the bearing surface. The sensing element is electrically connected to the patterned conductive circuit. The control unit is adjacent to the flexible sensing unit and is electrically connected to the patterned conductive circuit.

### CROSS REFERENCE TO RELATED APPLICATIONS

This Non-provisional application claims priority under 35 U.S.C. § 119(a) on Patent Application No. 202010385309.2 filed in People's Republic of China on May 9, 2020, the entire contents of which are hereby incorporated by reference.

### BACKGROUND OF THE INVENTION

### Field of the Invention

The invention relates to a monitoring device and, in particular, to a wearable physiologic state monitoring device.

### Descriptions of the Related Art

Timing after entering autumn, because the temperature difference between morning and evening becomes larger, the number of patients with respiratory tract infections is often increased. In a metropolis with a small space, people are close to each other, which will accelerate the transmission of respiratory vectors. Since the panic of Severe Acute Respiratory Syndrome (SARS) and avian influenza was imprinted in everyone's minds, the issue of respiratory infections has become a hot topic.

The respiratory tract is one of the windows of the human body to the external environment. In addition to providing the exchange of oxygen and exhaust gas, it also exposes the body to many pathogenic microorganisms. For the respiratory tract, it is generally divided into upper respiratory tract infection and lower respiratory tract infection.

Upper respiratory tract infection refers to infection of the nose, pharynx, throat, and sinuses by pathogens, including common cold, influenza, nasopharyngitis, acute tonsillitis, and laryngitis. The symptoms of upper respiratory tract infection are mainly nasal congestion, sneezing, runny nose, sore throat, cough, fever, headache, loss of appetite, and general fatigue.

Regarding the lower respiratory tract infection part, everyone is familiar with pneumonia. Pneumonia is an acute pulmonary air cell inflammation caused by bacteria or invisible virus, and it is still the top ten cause of death that threatens the lives of people. The main symptoms include high fever, cough, chest pain, etc., but less nasal congestion, sneezing, runny nose, sore throat, etc. The relatively severe symptoms often result in patients requiring hospitalization.

Whether it is upper respiratory tract infection or lower respiratory tract infection, if it can be detected early, it will enable patients to seek medical assistance as soon as possible, and help effectively control the condition that is not prone to deterioration.

With the popularity of wearable devices in recent years, it is limited to pedometer, heart rate, blood pressure or blood oxygen concentration monitoring. Accordingly, this application is to provide a wearable physiologic state monitoring device to enable the users to further monitor their own physiological state to achieve the purpose of early detection and early treatment.

### SUMMARY OF THE INVENTION

In view of the foregoing, the invention is to provide a wearable physiologic state monitoring device, which can be used in cooperation with clothing or accessories to enable the user to monitor his own physiological state while having a comfortable wearing experience.

To achieve the above, the invention provides a wearable physiologic state monitoring device, which includes a textile fabric, a flexible sensing unit, and a control unit. The textile fabric has a first surface and a second surface opposite to each other. The flexible sensing unit is joined to the first surface of the textile fabric and has a flexible substrate and a sensing element. The flexible substrate has a bearing surface, and a patterned conductive circuit is provided on the bearing surface. The sensing element is electrically connected to the patterned conductive circuit. The control unit is adjacent to the flexible sensing unit and is electrically connected to the patterned conductive circuit.

In one embodiment, the flexible sensing unit also has a flexible circuit board, which is arranged between the flexible substrate and the sensing element. The sensing element is arranged on the flexible circuit board and is electrically connected to the patterned conductive circuit on the flexible substrate through an electrode of the flexible circuit board.

In one embodiment, the sensing element is selected from a sensing electrode, a temperature sensing element, a strain sensing element, and combinations thereof

In one embodiment, the material of the patterned conductive circuit includes a conductive silver paste.

In one embodiment, the material of the flexible substrate is silicon, polyurethane (PU) or thermoplastic polyurethane (TPU).

In one embodiment, the control unit is connected to the textile fabric through a stud element, a bolt element or a bonding glue.

In another embodiment, the control unit may be disposed on the first surface or the second surface of the textile fabric.

In one embodiment, the flexible sensing unit is joined to the first surface of the textile fabric by a hot-pressing technology.

In one embodiment, a part of the sensing element is in contact with the bearing surface of the flexible substrate. In another embodiment, a part of the sensing element is in contact with the first surface of the textile fabric.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

The parts in the drawings are not necessarily drawn to scale, the emphasis instead being placed upon clearly illustrating the principles of at least one embodiment. In the drawings, like reference numerals designate corresponding parts throughout the various diagrams, and all the diagrams are schematic.

FIG. 1 is a schematic diagram showing a wearable physiologic state monitoring device according to an embodiment of the present invention.

FIG. 2 is a schematic diagram showing the flexible temperature sensing unit in the wearable physiologic state monitoring device in FIG. 1.

FIG. 3A is a schematic diagram showing a flexible resistive strain sensing unit in the wearable physiologic state monitoring device in FIG. 1.

FIG. 3B is a schematic diagram showing the flexible resistive strain sensing unit is joined to the clothes through the stud element.

FIG. 3C is a schematic diagram showing the flexible resistive strain sensing unit is joined to the clothes through the adhesive and the stud element.

FIG. 3D is a schematic diagram showing that the flexible resistive strain sensing unit is also electrically connected to the patterned conductive layer and the stretch element through the conductive connecting element.

FIG. 4 is a schematic diagram showing a flexible capacitive strain sensing unit in the wearable physiologic state monitoring device.

FIG. 5A is a schematic diagram showing the flexible ECG measuring unit in the wearable physiologic state monitoring device in FIG. 1.

FIG. 5B is a schematic diagram showing the patterned conductive circuit of the flexible ECG measuring unit according to another embodiment of the invention.

FIG. 6 is a schematic diagram showing the implementation of the control unit in the wearable physiologic state monitoring device on the second surface of the textile fabric.

FIG. 7 is a schematic diagram showing that the wearable physiologic state monitoring device of the present invention is an embodiment of the arm sleeve.

### DESCRIPTION OF THE PREFERRED EMBODIMENT

In the following description, this invention will be explained with reference to embodiments thereof. However, the description of these embodiments is only for purposes of illustration rather than limitation.

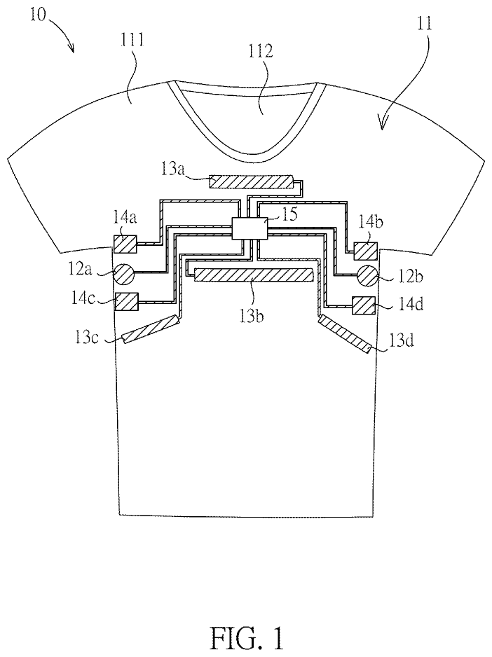

Please refer to FIG. 1, an embodiment of a wearable physiologic state monitoring device 10, which includes a textile fabric 11, two flexible temperature sensing units 12a-12b, and four flexible strain sensing units 13a-13d, four flexible ECG measuring units 14a-14d and a control unit 15.

The textile fabric 11 has a first surface 111 and a second surface 112 opposite to each other. The material of the textile fabric 11 can be textile fibers and fiber products, which are specifically represented by fibers, yarns, fabrics and their composites. Fibers include the natural fiber, the artificial fiber and the synthetic fiber, where the natural fiber can include cotton, wool, silk or hemp; the artificial fiber can be made of wood, cotton linters or natural cellulose from grass; the synthetic fiber mostly uses oil or natural gas as raw materials. In this embodiment, the specific performance of the textile fabric 11 can be clothes, pants, the arm sleeve, a corset, and a bra, which are various wearing articles that can be worn on the human body. In this embodiment, the specific performance of the textile fabric 11 as the clothing with elastic fabric is taken as an example, and the first surface 111 is close to the side of the human skin.

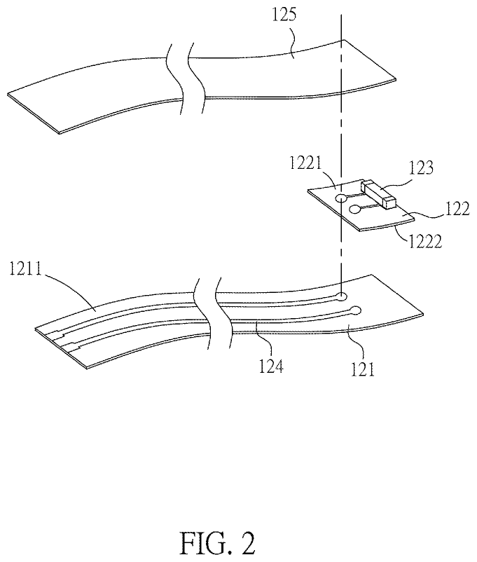

Please refer to FIG. 1 and FIG. 2 to illustrate the flexible temperature sensing units 12a-12b. The flexible temperature sensing units 12a-12b are respectively disposed on the positions of the clothes corresponding to the armpits of the human body. Taking the flexible temperature sensing unit 12a as an example, the flexible temperature sensing unit 12a has a flexible substrate 121, a flexible circuit board 122, a temperature sensing element 123, a patterned conductive circuit 124, and a flexible cover 125.

The flexible substrate 121 has a strip shape and has a bearing surface 1211. The flexible substrate 121 is bonded to the first surface 111 of the textile fabric 11 with the other surface opposite to the bearing surface 1211. The material of the flexible substrate 121 can be silicon, polyurethane (PU), or Thermoplastic Polyurethane (TPU). In the embodiment, TPU is taken as an example for illustration, which can be combined with the first surface 111 of the textile fabric 11 by the hot-pressing process.

The flexible circuit board 122 has a bearing surface 1221 and an bonding surface 1222 that are disposed oppositely. The bearing surface 1221 has a plurality of electrodes and a conductive circuit. The bonding surface 1222 can be bonded and fixed to the bearing surface 1211 of the flexible substrate 121 by the adhesive.

The temperature sensing element 123 is disposed on the bearing surface 1221 of the flexible circuit board 122. In the embodiment, the temperature sensing element 123 may be a thermistor or other electronic components that can change electrical output with temperature changes, and are electrically connected to the electrode through a solder ball, a bump, or a conductive adhesive.

The patterned conductive circuit 124 is mainly disposed on the bearing surface 1211 of the flexible substrate 121, and is electrically connected to the temperature sensing element 123 on the bearing surface 1221 of the flexible circuit board 122. Wherein, the flexible circuit board 122 may be provided with the electrode on the bonding surface 1222, and is electrically connected to the electrode of the bearing surface 1221 through a through via hole or a blind hole. Accordingly, the patterned conductive circuit 124 can be electrically connected to the temperature sensing element 123 through the electrode on the bonding surface 1222, the through via hole or the blind hole, and the electrode on the bearing surface 1221. In the embodiment, the material of the patterned conductive circuit 124 may include Conductive silver paste, which may be formed on the bearing surface 1211 of the flexible substrate 121 by screen process or direct printing.

The flexible cover 125 is approximately similar to the flexible substrate 121 in appearance. The flexible cover 125 disposes on the flexible substrate 121, and at least part of the flexible circuit board 122, the temperature sensing element 123, and the patterned conductive circuit 124 are covered between the flexible cover 125 and the flexible substrate 121. The flexible cover 125 is also made of the same material as the flexible substrate 121, and can be silicone, polyurethane or TPU. In the embodiment, the material of the flexible cover 125 is TPU as an example, which can be combined with the flexible substrate 121 by the hot-pressing process.

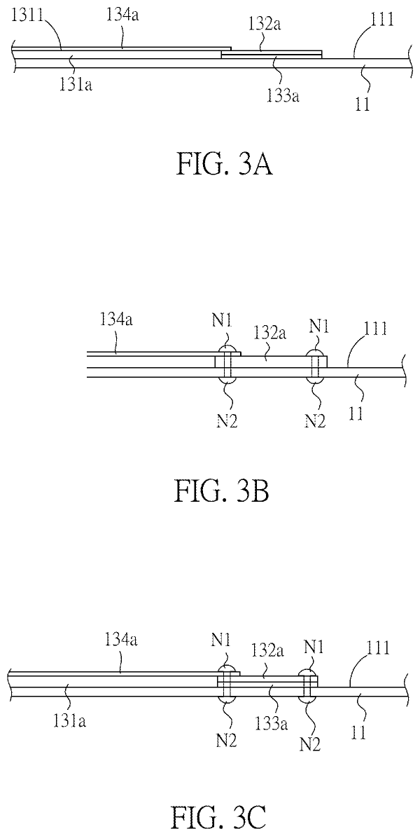

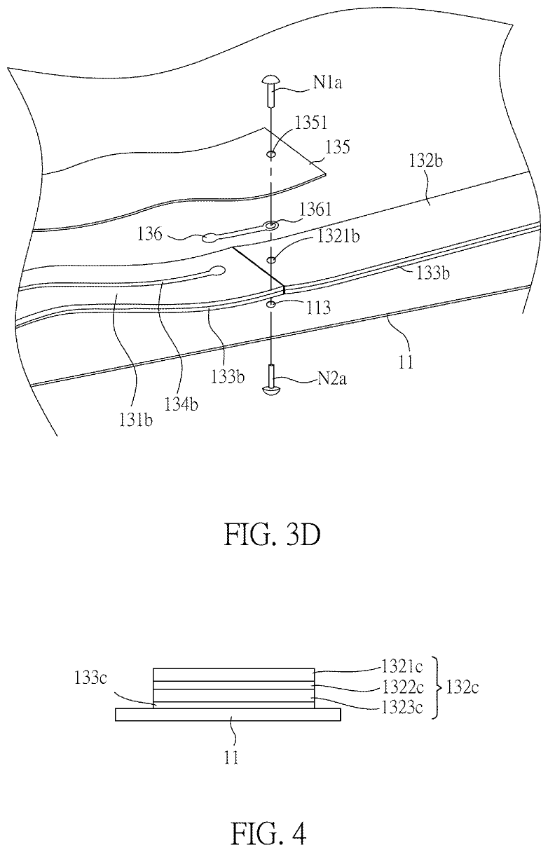

Then, please refer to FIG. 1, FIG. 3A to FIG. 3D and FIG. 4 to illustrate the flexible strain sensing unit 13a-13d. The flexible strain sensing unit 13a is arranged on the clothe corresponding to the upper side of the pectoralis of the human body, the flexible strain sensing unit 13b is arranged on the clothe corresponding to the lower side of the pectoralis of the human body, and the flexible strain sensing unit 13c-13d is provided on the clothes corresponding to the intercostal muscles of the human body. The physiological parameters about the human breathing state can be obtained by monitoring the specific musculature. It is to be noted, the flexible strain sensing unit can be a flexible resistive strain sensing unit or a flexible capacitive strain sensing unit, which will be described separately below.

Please refer to FIG. 1 and FIG. 3A, the flexible strain sensing unit 13a is the flexible resistive strain sensing unit, which has a flexible substrate 131a, a stretch element 132a, a combining element 133a, and a patterned conductive circuit 134a.

The flexible substrate 131a is a strip shape and has a bearing surface 1311. The flexible substrate 131a is bonded to the first surface 111 of the textile fabric 11 with the other surface opposite to the bearing surface 1311. The patterned conductive circuit 134a is formed on the bearing surface 1311 of the flexible substrate 131a by the screen-printing process or directly printing. The flexible substrate 131a has the same structure, materials, and bonding methods as the flexible substrate 121 described above that includes connecting by direct hot-pressing process, connecting through the adhesive, connecting through the stud element, bolt element, and combinations thereof.

The stretch element 132a is formed by using silicone as the base material and mixing the conductive particles in the base material. In other embodiments, silicone can also be replaced with other elastic materials. The stretch element 132a is electrically connected to the patterned conductive circuit 134a on the flexible substrate 131a. The combining element 133a is, for example, the adhesive, so that the stretch element 132a is fixed to the first surface 111 of the textile fabric 11 by gluing. The adhesive is, for example, hot-melt adhesive (HMA), which can fix the stretch element 132a to the clothes by hot-pressing process. It is to be noted, in addition to the form of glue, the combining element can also be the combining element in the form of locking or snapping.

Other embodiments of the combining element 133a are, for example, a stud element or a bolt element. Please refer to FIG. 3B, take the stud element as an example. The stud element has a male stud N1 and a female stud N2. In the embodiment, the male stud N1 and the female stud N2 run through the flexible substrate 131a, the stretch element 132a, and the textile fabric 11 to be combined with each other, so that the stretch element 132a is fixed on the textile fabric 11. It is to be noted, in the embodiment, the stud element can be conductive and can be used for electrical conduction. In addition, the combining element 133a can also have the above-mentioned adhesive and the stud element as shown in FIG. 3C.

In addition, please refer to FIG. 3D, in another embodiment, the flexible strain sensing unit 13b has a flexible substrate 131b, a stretch element 132b, a combining element 133b, a patterned conductive circuit 134b, a flexible cover 135, and a conductive connecting element. 136. Among them, the flexible substrate 131b and the stretch element 132b are glued and fixed to the first surface 111 of the textile fabric 11 through the combining element 133b. The patterned conductive circuit 134b is electrically connected to the stretch element 132b through the bridging of the conductive connecting element 136. The flexible cover 135 is bonded to the flexible substrate 131b, and covers the patterned conductive circuit 134b, the conductive connecting element 136, and part of the stretch element 132b to protect it. In addition, the male stud N1a and the female stud N2a of the stud element can pass through the through hole 1351 of the flexible cover 135, the through hole 1361 of the conductive connecting element 136, the stretch element 132b, and the combining element 133b. The through hole 1321b of the textile fabric 11 and the through hole 113 of the textile fabric 11 are combined with each other to be fixed.

The flexible strain sensing unit can be the flexible capacitive strain sensing unit in addition to the above-mentioned resistive mode. For a brief description, please refer to FIG. 4, the flexible capacitive stretch element 132c has a first conductive layer 1321c, an insulating layer 1322c, and a second conductive layer 1323c, which are layered. The first conductive layer 1321c and the second conductive layer 1323c are respectively formed by using silicone as the base material and mixing the conductive particles in the base material. The flexible capacitive stretch element 132c can also be combined with the textile fabric 11 through a combining element 133c. The electrical conduction mode of the flexible capacitive stretch element 132c can be electrically connected to the first conductive layer 1321c and the second conductive layer 1323c by disposing a patterned conductive pattern on both sides of the flexible substrate.

In addition to the resistive and capacitive types described above, the flexible strain sensing unit can also be changed in other forms. The main feature is that its electrical characteristics will change with its length.

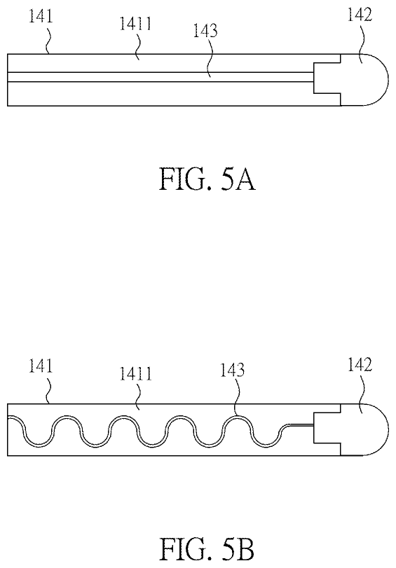

Then, please refer to FIG. 1 and FIG. 5A to explain the flexible ECG measuring units 14a-14d, where the flexible ECG measuring units 14a-14b are respectively disposed on the clothes corresponding to the left and right pectoralis muscles of the human body, and the flexible ECG measuring units 14c-14d are respectively disposed on the clothes corresponding to between the ribs on the left side of the human body or between the ribs on the right side of the human body. To further explain, the flexible ECG measuring units 14a-14b can be located at the same height between the arm and chest; the flexible heart sensor units 14c-14d can be located between the first rib and the third from last rib. Regarding the structure of the flexible ECG measuring unit, the flexible ECG measuring unit 14a is taken as an example. The flexible

ECG measuring unit 14a has a flexible substrate 141, a sensing electrode sheet 142, and a patterned conductive circuit 143.

The flexible substrate 141 has a bearing surface 1411, and is bonded to the first surface 111 of the textile fabric 11 with the other surface opposite to the bearing surface 1411. The flexible substrate 141 has the same structure, materials, and bonding methods as the flexible substrate 131a described above that includes connecting by direct hot-pressing process, connecting through the adhesive, connecting through the stud element, bolt element, and combinations thereof.

The sensing electrode sheet 142 is disposed on the bearing surface 1411 at one end of the flexible substrate 141. A part of the sensing electrode sheet 142 is in contact with the bearing surface 1411, and a part of the sensing electrode sheet 142 protrudes from the flexible substrate 141. The sensing electrode sheet 142 can be joined by the adhesive and fixed to the bearing surface 1411 of the flexible substrate 141. In other embodiments, the sensing electrode sheet 142 can also be completely disposed on the bearing surface 1411 of the flexible substrate 141.

The patterned conductive circuit 143 is disposed on the bearing surface 1411 of the flexible substrate 141, and is electrically connected to the sensing electrode sheet 142. The material of the patterned conductive circuit 143 may include a conductive silver paste, which may be formed on the bearing surface 1411 of the flexible substrate 141 by the screen-printing process or directly printing. It is to be noted, for factors such as impedance matching, structural strength, or circuit layout optimization, the patterned conductive circuit 143 can be in a serpentine-like S-shape (such as FIG. 5B) in addition to being straight. In addition, the patterned conductive layer mentioned in the present invention may also have a serpentine-like S-shaped design as shown in FIG. 5B. The patterned conductive circuit or the patterned conductive layer of the serpentine-like S-shaped design can have different curves and lengths according to the impedance matching design. To further illustrate, the curves of each segment of the S-like design can also be different. In addition, the serpentine-like S-shaped design also helps maintain certain electrical conductivity after the stretching process.

Please refer to FIG. 1, the control unit 15 is disposed on the clothes corresponding to the position of the human chest. The control unit 15 can be joined to the clothes by hot melt glue, fastening element or devil felt, and is electrically connected with the flexible temperature sensing units 12a-12b, the flexible strain sensing units 13a-13d and the flexible ECG measuring units 14a-14d. In particular, in the flexible temperature sensing units 12a-12b, the flexible strain sensing units 13a-13d and the flexible ECG measuring units 14a-14d, the end of the patterned conductive circuit may have an electrode, which may be the so-called gold finger or edge connector. The so-called gold finger or edge connector is the metal terminal or pin on the circuit board. The electrode can be electrically connected by plugging into the socket of the control unit 15. The socket of the control unit 15 may be a zero insertion force (ZIF) socket. To further illustrate, the patterned conductive circuit and each sensing unit can also be electrically connected through the zero insertion force socket.

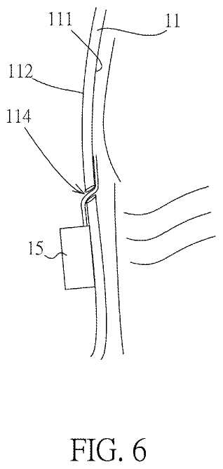

In this embodiment, the control unit 15 is joined to the first surface 111 of the textile fabric 11 by hot melt glue. In other embodiments, as shown in FIG. 6, it is showing the clothe is on the human body. The control unit 15 can also be joined to the second surface 112 of the textile fabric 11 by hot melt adhesive. As shown in FIG. 6, the textile fabric 11 around the control unit 15 needs to be provided with the through hole 113 so that the flexible temperature sensing units 12a-12b, the flexible strain sensing units 13a-13d and the flexible substrate of the flexible ECG measuring units 14a-14d pass through the first surface 111 to the second surface 112 and is electrically connected to the control unit 15.

The control unit 15 can include functions such as calculation, storage, and communication to perform subsequent processing on the signals sensed by the flexible temperature sensing units 12a-12b, the flexible strain sensing units 13a-13d, and the flexible ECG measuring units 14a-14d. Taking the strain sensing as an example, the flexible strain sensing units 13a-13d can measure the change of the stretch length of the stretch element during a unit time. The breathing rate of the user can be obtained after the change of the stretch length after a differential operation; the breathing intensity of the user can be obtained after the second differential operation; and the breathing volume of the user can be obtained after an integral operation. According to the above, it is possible to timely send out notifications to remind the user when an abnormal breathing rate or an abnormal breathing intensity occurs in the user through the change of the data.

The control unit 15 may communicate externally through a communication unit. The technology used by the communication unit may include Radio frequency identification (RFID), Near field communication (NFC), Zigbee, Narrow band internet of things (NB IoT), LoRa, Sigfox, Bluetooth Or Wi-Fi. Through the communication unit described above, the control unit 15 can transmit data to the electronic device designated by the user, such as but not limited to a mobile communication device, a terminal arithmetic device, or a cloud database. In the embodiment, an antenna of the communication unit can be formed on the printed circuit board or the flexible circuit board by screen-printed to be integrated into the control unit 15.

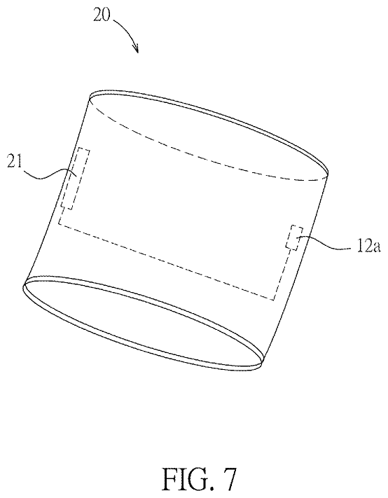

Please refer to FIG. 7, the above communication function can also be a single communication unit and operate independently. FIG. 7 is illustrated with an arm sleeve 20 as an example, which can be worn on the arm of a human body. The flexible temperature sensing unit 12a as described above can be arranged on the inner side of the arm sleeve 20, and a communication unit 21 is electrically connected to and arranged adjacent to the flexible temperature sensing unit 12a. The communication unit 21 can be paired with a smart mobile device, such as a mobile phone, and transmit the temperature information sensed by the flexible temperature sensing unit 12a to the smart mobile device. In other embodiments, the communication unit in the implementation aspect of the arm sleeve can also be replaced with the control unit mentioned above, and the flexible temperature sensing unit can also be replaced with other the flexible sensing unit. In addition, in other embodiments, the antenna of the communication unit can be formed on the flexible substrate by the screen-printing technology, and then joined to the arm sleeve by the hot-pressing technology. The electrical connection between the communication unit and the flexible sensing unit can be selected through the above-mentioned various connection methods.

As mentioned above, the wearable physiologic state monitoring device disclosed in the present invention combines various flexible sensing units with special technical structures on the surface of the soft textile fabric. Since the sensing unit has sufficient flexibility and good electrical characteristics, it is easy to be integrated into daily necessities such as clothes or textiles. In this way, it can be ensured that the physiological signals of the human body are monitored in real time, so that abnormalities in the body can be detected early. In addition, when the sensing unit has the flexible substrate and the flexible cover, it can be ensured that the sensing unit can be cleaned with the textile fabric at the same time, which can further improve the convenience of the user.

The above embodiments merely give the detailed technical contents of the present invention and inventive features thereof, and are not to limit the covered range of the present invention. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A wearable physiologic state monitoring device, comprising:
a textile fabric, which has a first surface and a second surface opposite to each other;
a flexible sensing unit, which is joined to the first surface of the textile fabric, comprising:
a flexible substrate, which has a bearing surface, and a patterned conductive circuit is provided on the bearing surface; and
a sensing element, which is electrically connected to the patterned conductive circuit disposed on the bearing surface of the flexible substrate; and
a control unit, which is adjacent to the flexible sensing unit and is electrically connected to the patterned conductive circuit.

2. The wearable physiologic state monitoring device of claim 1, wherein the flexible sensing unit further comprising:
a flexible circuit board, which is disposed between the flexible substrate and the sensing element, where the sensing element is disposed on the flexible circuit board and is electrically connected to the patterned conductive circuit on the flexible substrate through an electrode of the flexible circuit board.

3. The wearable physiologic state monitoring device of claim 1, wherein the sensing element is selected from a sensing electrode, a temperature sensing element or a strain sensing element.

4. The wearable physiologic state monitoring device of claim 1, wherein the material of the patterned conductive circuit includes a conductive silver paste.

5. The wearable physiologic state monitoring device of claim 1, wherein the material of the flexible substrate is silicon, polyurethane (PU) or thermoplastic polyurethane (TPU).

6. The wearable physiologic state monitoring device of claim 1, wherein the control unit is connected to the textile fabric through a stud element, a bolt element or a bonding glue.

7. The wearable physiologic state monitoring device of claim 1, wherein the control unit is disposed on the first surface or the second surface of the textile fabric.

8. The wearable physiologic state monitoring device of claim 1, wherein the flexible sensing unit is joined to the first surface of the textile fabric by a hot-pressing technology.

9. The wearable physiologic state monitoring device of claim 1, wherein a part of the sensing element is in contact with the bearing surface of the flexible substrate.

10. The wearable physiologic state monitoring device of claim 1, wherein a part of the sensing element is in contact with the first surface of the textile fabric.
